(19) United States
(12) Patent Application Publication
(10) Pub. No.: US 2009/0269330 A1
(43) Pub. Date: Oct. 29, 2009

(54) FATTY ACID TRIGLYCERIDES FOR MAKING BIOCOMPATIBLE COATINGS

(75) Inventors: Pierre Jacobs, Gooik (BE); Bert Sels, Balen (BE); Ivan De Scheerder, Herent (BE)

Correspondence Address: CLARK & ELBING LLP, 101 FEDERAL STREET, BOSTON, MA, 02110, US

(73) Assignee: ZISCOAT NV, Heverlee (BE)

(21) Appl. No.: 12/300,807

(22) PCT Filed: May 18, 2007
(86) PCT No.: PCT/EP07/04464

§ 371 Date: Nov. 14, 2008

Related U.S. Application Data
(60) Provisional application No. 60/747,495, filed on May 17, 2006.
(60) Provisional application No. 60/803,788, filed on Jun. 2, 2006.

(30) Foreign Application Priority Data
Jun. 2, 2006 (GB) 0610794.0

(51) Int. Cl. C09D 4/00; A61K 39/395; A61F 2/06; A61M 39/00; A61M 25/00; A61B 17/08

(52) U.S. Cl. 424/130.1; 106/243; 623/1.42; 604/8; 604/265; 606/151

(57) ABSTRACT

The invention relates to a mixture (a) of fatty acid triglycerides having a slip melting point above 500 C, the fatty acid composition of said mixture (a) comprising at least two different fatty acids and comprising from 20 to 95% by weight of saturated fatty acids and 80 to 5% by weight of unsaturated fatty acids, and wherein at least one fatty acid triglyceride (a1) from said mixture (a) bears the same saturated fatty acid residue on each position of the glycerol moiety and corresponds to at least 10% by weight of said mixture (a). This mixture is useful in the coating of medical devices to improve bio-compatibility, stability, and drug release.

### FIELD OF THE INVENTION

The present invention relates to fatty acid triglyceride mixtures for the controlled delivery of bioactive substances and/or for the biocompatible coating of medical devices, for example implantable devices.

### BACKGROUND OF THE INVENTION

The administration of anti-restenotic therapeutic agents using systemic (oral or intravenous) administration after dilatation of narrowed lumina (for example, of a coronary arterial atherosclerotic narrowing) has often provided disappointing therapeutic outcome in clinical trials due to the fact that the local concentration of the therapeutic agent where its effect is required is too low and due to the systemic side effects of the therapeutic agent when higher doses are administered. For this reason, therapeutic agents have been administered locally, to the organ to be treated. For instance, in the treatment of coronary stenoses, therapeutic agents have been injected into the vessel wall using special catheters. Disadvantages of this approach are still the limited efficiency of the so-called local treatment (less than 5% of the administered therapeutic agent reaches the target organ) and the increased damage to the target organ due to the local drug administration.

Another approach is the coverage of an implantable device, such as an endoluminal prosthesis, with a coating of polymers comprising the therapeutic agent (see, for instance, EP-A-0,623,354). The disadvantages of this method have been shown to be the limited drug capacity of the coating and the too fast release of the therapeutic agent because of the large contact area. Furthermore, most polymers need a quite aggressive polymerisation step that can result in the inactivation of the therapeutic agent and most polymers are not very biocompatible and may induce a foreign body inflammatory response, resulting in even more hyperplasia and restenosis.

WO 03/03961 describes the use of biological oil based stent coatings. An improved biocompatibility is demonstrated and the need for an aggressive polymerisation step is avoided.

EP-1,576,970 teaches the use of chemically hardened fat coatings for implantable medical devices, whereby the coating is stabilised by hydrogenation of the double bonds. This approach provides a chemically more stable product, less prone to oxidation, leading to an improved shelf-life and furthermore a slow breakdown of the coating after implantation in a coronary stent model. In an in vitro model, a direct relationship between the mass loss of the coating and the degree of hydrogenation was demonstrated. More particularly, hydrogenation may be obtained by the methods of U.S. Pat. No. 6,229,032 and EP-0,917,561 in order to limit formation of trans bonds.

However there is still a need in the art for improving the chemical stability and the plasticity of coatings for implantable medical devices, as well as the long-term storage capacity of medical devices and pharmaceutical preparations coated with materials derived from natural oils and fats. There is also a need in the art for improving the resistance of the coating against mass loss in vivo.

### SUMMARY OF THE INVENTION

The present invention provides alternative coatings for medical devices such as implantable medical devices, e.g. stents. An advantage of the present invention is an increased stability and reproducibility resulting in a reduced degeneration of the coating. Another advantage is that it allows for controlled release of a bioactive agent.

In a first aspect, the present invention provides mixtures (a) of fatty acid triglycerides, wherein the slip melting point of said mixture (a) is above 50° C., the fatty acid composition of said mixture (a) comprises at least two different fatty acids and comprises, e.g. consists of from 20 to 95% by weight of saturated fatty acids and 80 to 5% by weight of unsaturated fatty acids, and wherein at least one fatty acid triglyceride (a1) from said mixture (a) bears the same saturated fatty acid residue on each position of the glycerol moiety and corresponds to at least 10% by weight of said mixture (a).

In a second aspect, the present invention provides bio-compatible coating compositions for a medical device comprising fatty acid triglyceride mixtures according to the first aspect of the invention.

In a third aspect, the present invention provides a process for preparing a mixture (a) of fatty acid triglycerides according to the first aspect of the invention and comprising the steps of:
providing a fatty acid triglyceride (a1) bearing the same saturated fatty acid residue on each position of the glycerol moiety,
providing one or more fatty acid triglycerides other than (a1), and
interesterifying said fatty acid triglyceride (a1) with said one or more other fatty acid triglycerides,
   wherein the amount of (a1) before the interesterification step is greater than 10% by weight of the total amount of fatty acid triglycerides, wherein the fatty acid composition of one or more fatty acid triglycerides other than (a1) comprises at least two different fatty acids and contains from 20 to 95% of saturated fatty acids and from 80% to 5% of unsaturated fatty acids, and wherein the interesterification step is effected in such a way that the resulting mixture comprises at least 10% by weight of (a1).

In a fourth aspect, the present invention provides medical devices comprising, on at least part of a surface thereof, at least one coating having a composition according to the second aspect of the invention. In a fifth aspect, the present invention provides a method for making such coated medical devices.

### BRIEF DESCRIPTION OF THE DRAWINGS

FIG. 1 shows representative photomicrographs (magnification 25 for A and B; magnification 100 for C and D) of a vessel segment stented with stents coated with comparative hydrogenated triglycerides (A and C) or with transesterified triglycerides according to an embodiment of the present invention (B and D) 7 days after implantation in the coronary arteries of pigs.

FIG. 2 shows (E, left part of the figure) the injury score and (F, right part of the figure) the inflammation score in the coronary arteries of pigs for the comparative hydrogenated triglycerides coatings referred as “Ciscoat”, and the transesterified triglyceride coatings according to an embodiment of the present invention referred as “TRANSEST”.

FIG. 3 shows absolute taxol release from a transesterified triglyceride coating according to an embodiment of the present invention.

FIG. 4 shows percentage amount of taxol release from a transesterified triglyceride coating according to an embodiment of the present invention.

FIG. 5 shows the high performance liquid chromatography refractive index diagrams of a comparative triglyceride mixture.

FIG. 6 shows the high performance liquid chromatography refractive index diagram of a representative embodiment of a triglyceride mixture according to the present invention.

### DEFINITIONS

In the context of this invention, transesterification with regard to a triglyceride refers to the fact that at least one of the fatty acid chains originally present in said triglyceride is replaced by another fatty acid chain. Depending on whether the fatty acid chain originates from a mono acidic or di-acidic fat or acyl ester or from another triglyceride, reference is made to either interesterification or transesterification.

As used herein, unless otherwise stated, the term “controlled release” is a synonym for sustained-release, extended-release or prolonged-release, and refers to providing a steadier level of a biologically active agent in the bloodstream, and/or in the surrounding tissue of an implant (in particular in the vascular wall), of a mammal, in particular a human being, over a defined period of time. This defined period of time can range from a few hours to two months since “controlled release” is dependent on the therapeutic window of the biologically active agent that is used or of the biologically active agents that are used. More specifically “controlled release” can refer to the release of 50 to 95% of said bio-active agent within 4 to 24 hours.

“Controlled release” can also refer to the release of 15 to 50% of said bio-active agent within a period of time of at least 10 hours, 20 hours or even more than 50 hours, and/or the release of 75% of said bio-active agent within a period of time of at least 1 week, more than 2 weeks or even more than 5 weeks.

As used herein, unless otherwise stated, the term “fatty acid” conventionally refers to a saturated or ethylenically mono- or poly-unsaturated monocarboxylic acid with a preferably non-branched aliphatic chain having from 4 to 26 carbon atoms.

### DETAILED DESCRIPTION OF THE INVENTION

The present invention relates to novel transesterified fatty acid triglyceride mixtures useful in the coating of medical devices and pharmaceutical preparations.

Using controlled transesterification according to an aspect of the present invention, it is possible to produce stable and reproducible coatings, which also have a surprisingly good plasticity resulting in improved coating characteristics.

This finding provides the unexpected advantage that by using this material as for example a stent coating or a coating for medicine pills, storage is much less sensitive to potential inactivation due to for example melting during storage.

With the transesterified triglyceride based coatings according to an embodiment of this invention, having melting points and/or slip melting points above 50° C., it is possible to obtain reproducible, homogeneous, and smooth coatings.

These transesterified triglyceride coatings also exhibit a very good resistance against mass loss in an in-vitro model. Also in vivo, in a coronary swine model, disappearance of the coating over time, evaluated by serial fat stains is slow. Combined with a much more reproducible and slower in-vitro drug release, this has proven to be a very reliable coating, particularly for medical devices.

Furthermore, using the methods of transesterification of the present invention, it is possible to provide coatings of transesterified triglycerides which do not contain three double bonds on the same chain, such that chemical stability of these coatings is also superior compared to previous coatings, since the coatings are less prone to oxidation.

The transesterified triglycerides according to an embodiment of the present invention can be obtained by an interesterification reaction on a mixture of two or more triglycerides, preferably a partial interesterification, for example by stopping the interesterification reaction at a stage wherein a substantial amount of at least one of the starting triacyglycerol is still present, most preferably this remaining starting triacylglycerol is a glycerol bearing the same saturated fatty acid residue on each position.

The fatty acid composition of the transesterified triglycerides according to an embodiment of the present invention is an important factor but can vary in a broad range, while still providing advantages over the mixtures of the prior art. In general, the fatty acid composition of the transesterified triglycerides comprises from 20 to 95% by weight of saturated fatty acid, preferably from 30 to 80% by weight, most preferably from 40 to 60% by weight. Accordingly the fatty acid composition of the transesterified triglyceride according to an embodiment of the present invention usually comprise from 80 to 5% by weight of unsaturated fatty acid, preferably from 70 to 20% by weight, most preferably from 60 to 40% by weight.

The triglyceride composition of the transesterified triglycerides according to an embodiment of the present invention can also be broadly varied, provided that the slip melting point of the composition is above 50° C., preferably below 100° C., most preferably below 70° C., while keeping good plasticity and improved coating characteristics. This can be obtained by interesterification of a mixture having a fatty composition as described herein above, wherein after the interesterification is stopped, at least one of the fatty acid triglyceride has the same saturated fatty acid residue on the three positions of the glycerol moiety and is present in at least 10% by weight of the triglyceride mixture, for instance at least 15% by weight, or at least 20% by weight.

Certain methods of obtaining transesterified triglycerides are already known in the art. The specific process used in an embodiment of the present invention is described in the Examples below. The most common method is chemical transesterification, wherein the reaction is catalysed by the addition of an acid, such as toluenesulfonic acid, or a base, such as sodium methoxide. Typically a drying and degassing process precedes transesterification so as to ensure that all moisture is reliably removed. Optimally, the catalyst is finely dispersed to allow the reaction to proceed smoothly. The reaction is generally performed in the temperature range between 70° C. and 120° C. The reaction time may be less than 1 hour, for instance 30 minutes, and up to 24 hours. The catalyst is preferably inactivated after completion of the reaction. Inactivation of the catalyst is typically achieved by adding water, dilute mineral acid (when the catalyst is a base), or else a mixture of water and carbon dioxide.

Transesterification can be either controlled or uncontrolled (e.g. random). Typically, in controlled transesterification, the thermo-dynamic equilibrium state is used as the starting point and this is deliberately interfered with by crystallising high-melting and sparingly soluble triglycerides which are present or are formed by transesterification and removing these from the equilibrium.

Alternatively transesterified triglycerides can be obtained using enzymes as catalysts. This process is generally applied in the case of transesterification starting from natural materials such as from palm oil or coconut oil. Examples of catalysts used in enzymatic trans-esterification are 1,3 specific lipases (such as from Novozyme) well known in the art.

The triglycerides used in the context of the present invention can be oils or fats, more particularly biological oils or fats such as, but not limited to, vegetable oil or fish oil.

According to a particular embodiment, preferably purified triglycerides are used as starting materials to produce the transesterified fatty acid triglycerides for the coatings of the present invention. The fatty acid chains of these preferably purified base triglycerides can be saturated or unsaturated.

Suitable saturated fatty acids can be, but are not limited to, those selected from the group consisting of butyric acid (C₄), valeric acid (C₅), caproic acid (C₆), enanthic acid (C₇), caprylic acid (C₈), pelargonic acid (C₉), capric acid (C₁₀), lauric acid (C₁₂), myristic acid (C₁₄), pentadecanoic acid (C₁₅), palmitic acid (C₁₆), margaric acid (C₁₇), stearic acid (C₁₈), arachidic acid (C₂₀), behenic acid (C₂₂), lignoceric acid (C₂₄), and cerotic acid (C₂₆).

Suitable unsaturated fatty acids can be, but are not limited to, those selected from the group consisting of oleic acid, erucic acid, nervonic acid, linoleic acid, γ-linolenic acid, arachidonic acid, α-linolenic acid, eicosapentanoic acid (EPA), docosahexanoic acid (DHA), oleic acid, palmitoleic acid and myristoleic acid. Preferentially, the fatty acids do not contain trans-double bonds.

According to another embodiment of the present invention, the transesterified triglycerides comprise triglycerides, i.e. a triglyceride with one fatty acid chain and two short acid chains (e.g. acetoyl or propanoyl), such as 1,3-diacetopalmitin, or a triglyceride with two fatty acid chains and one short acid chain (e.g. acetoyl or propanoyl).

More particularly, transesterified triglycerides comprising a limited number of double bonds (e.g. not more than 3 ethylenic unsaturations per fatty acid) are envisaged. In one embodiment, the fatty acid chains of the transesterified triglycerides of the present invention have a length between 4 and 26 carbon atoms, preferably from 8 and 24 carbon atoms, more preferably from 12 and 20 carbon atoms. Particularly a coating according to an embodiment of the present invention comprises transesterified fatty acids of which the fatty acid chains are selected from the group of lauric fatty acid, myristic fatty acid, palmitic fatty acid, stearic fatty acid, oleic fatty acid linoleic fatty acid and arachidic fatty acid chains. In one embodiment, the coatings of the present invention comprise transesterified triglycerides consisting of fatty acids chains, each of which have less than three double bonds. According to a further embodiment, the transesterified fatty acids of the coating of the invention comprise only saturated and/or mono-unsaturated fatty acid chains. According to an alternative embodiment, the transesterified fatty acids comprise oleic, linoleic and stearic fatty acid chains. According to another embodiment, the transesterified fatty acids comprise palmitic, oleic and linoleic fatty acid chains. According to one embodiment the transesterified fatty acids are obtained by random transesterification of tricarboxylic acid glyceryl esters comprising three identical carboxylic acid chains (triolein, trilinolein, tristearin, etc.), whereby the composition is characterised by the relative ratio of the starting products. According to this specific embodiment, upon partial interesterification, the fatty acid triglycerides mixture obtained still comprises two or more tricarboxylic acid glyceryl esters bearing three identical carboxylic acid chains. For example, the mixture still contains at least 10% by weight of both a fatty acid triglyceride bearing the same saturated fatty acid and at least 6% by weight of a fatty acid triglyceride bearing the same unsaturated fatty acid.

The fatty acid chains of said transesterified triglycerides can comprises, e.g. consist of between 20-40% by weight linoleic acid, between 20-40% by weight stearic acid and between 20-40% by weight oleic acid. When using a triolein/tristearin/trilinolein triglyceride combination, a reproducible stable coating with a melting point of around 65° C. was obtained, independent of the triglyceride ratios used. The slip melting point of this coating was 62° C. This finding provides the unexpected advantage that by using this material as for example a stent coating or medicine pills, storage is much less sensitive to potential inactivation due to for example melting during storage.

According to another aspect, the invention provides a process for preparing mixtures of fatty acid triglycerides according to the first aspect of the invention and comprising the steps of:
providing a fatty acid triglyceride (a1) bearing the same saturated fatty acid residue on each position of the glycerol moiety,
providing one or more fatty acid triglycerides other than (a1), and
interesterifying said fatty acid triglyceride (a1) with said one or more other fatty acid triglycerides,
   wherein the amount of (a1) before the interesterification step is greater than 10% by weight of the total amount of fatty acid triglycerides, the fatty acid composition of the provided fatty acid triglycerides contains from 20 to 95% of saturated fatty acids and from 80% to 5% of unsaturated fatty acids, and wherein the interesterification step is carried out such as the resulting mixture comprise at least 10% by weight of (a1).

In order to obtain a resulting mixture containing at least 10% by weight of (a1), the interesterification step or partial inter-esterification step (depending on the type and amount of starting materials) can be carried for a limited time, which is, as understood by the person skilled in the art, directly correlated to the temperature at which said interesterification takes place and the type and amount, if any, of catalyst present.

According to a further aspect, the present invention provides a matrix for pharmaceutical compositions like structures, sprays, dermal solutions, particles, pills and solutions for medical use.

The coating compositions according to an embodiment of the present invention contain between 10-100% by weight transesterified fatty acid triglycerides. Where a bioactive agent is included within the coating, the transesterified triglycerides can make up between 10-90% by weight of the coating. However, other ratios are envisaged, for example coatings wherein the amount of said bioactive agent compound is from 0.1% to 50% by weight.

According to one aspect, transesterified triglycerides are used to coat or to cover medical devices. This medical device can be for example an intraluminal prosthesis, a stent, a shunt, a catheter, a local drug delivery device, surgical wires or clips.

In a particular embodiment the present invention relates to an intraluminal device, in particular an intraluminal prosthesis, stent, shunt, catheter or local drug delivery device, provided with at least one transesterified triglyceride based coating, which sticks to the intraluminal device. This coating does not need an aggressive polymerisation step, is chemically stable and is biocompatible. It has been found rather surprisingly that these transesterified triglyceride coatings adhere well to the intraluminal device so that most of the coating remains on the intraluminal device when inserting it into the lumen. Due to the selection of the triglycerides, the coating reduces the foreign body inflammatory response induced by the intraluminal device.

According to one embodiment, the coating of transesterified triglycerides comprises a therapeutic agent. In a particular embodiment the present invention therefore provides a new intraluminal device which is provided with a transesterified triglyceride coating which enables to obtain a sustained local release of one or more therapeutic agents. To achieve this object, the intraluminal device according to an embodiment of the present invention is characterised in that the matrix which comprises the therapeutic agent is formed by transesterified triglycerides.

In particular embodiments of the invention the coatings are used for the controlled delivery of bioactive agents. The term bioactive agents includes therapeutic or prophylactic compounds, compounds which, in combination with one or more other compounds, are administered separately or present locally have a therapeutic or prophylactic effect, compounds for use in identification purposes etc. . . .

According to one embodiment, the coating slows down the release of the therapeutic agent once inserted in the body lumen.

Instead of being a component of the coating, the therapeutic agent may also be chemically combined with the coating by any chemical combination technique. The therapeutic agent may for example be chemically bound to the fatty acid groups or to the glycerol group.

In a device according to an embodiment of the present invention, the therapeutic agent may also be mixed with the transesterified triglycerides. When soluble in the coating substance, the therapeutic agent can be dissolved therein or, when it is not soluble in the coating substance, it can be dispersed therein, more particularly emulsified or suspended depending on the fact whether the therapeutic agent is a liquid or a solid.

The therapeutic agent may be selected from the group of neointimal hyperplasia inhibiting drugs, platelet inhibiting drugs, smooth muscle cells dedifferentiation inhibiting drugs, smooth muscle cell proliferation inhibiting drugs, and smooth muscle cell migration inhibiting drugs.

More specific the therapeutic agent may be selected from the group of drugs consisting of vinblastine, sirolimus, mitoxantrone, tacrolimus, paclitaxel, cytochalasin, latrunculin, and everolimus, and the precursors, analogues and/or derivatives of these drugs.

It can also be selected from the group consisting of deferoxamine, geldanamycin, nigericin, penitrem, paxilline, verruculogen, KT5720, KT5823, Anisomycin, chelerythrine chloride, genistein, parthenolide, trichostatin A, T2 toxin, Zearalenone, Interferon, epithalon-D, Ca-ionophore, 4 bromo Ca Ionophore, Aflatoxins, aphidicolin, brefeldin A, cerulenin, chromomycin A3, citrinin, cyclopiazonic acid, forsokolin, fumagillin, fumonisins B1, B2, hypericin, K252, mycophenolic acid, ochratoxin A, and oligomycin or further from the group consisting of mycophenolic acid, mycophenolate mofetil, mizoribine, methylprednisolone, dexamethasone and other corticosteroids, Certican™, Tritolide™, Methotrexate™, Benidipine™, Ascomycin™, Wortmannin™, LY 294002, Camptothecin™, Topotecan™, hydroxyurea, cyclophosphamide, cyclosporin, daclizumab, azathioprine, Gemcitabine™, and precursors, derivatives and analogues of these drugs. As therapeutic agents, genes, coding for certain substances (proteins), having either anti-thrombotic, anti-inflammatory, anti-mitotic anti-angiogenesis and/or anti-restenotic action, can be used as well.

The therapeutic agent may have different effects and may in this respect be selected amongst immunosuppressants, anti-inflammatories, anti-proliferatives, anti-migratory agents, anti-fibrotic agents, proapoptotics, calcium channel blockers, anti-neoplastics, antibodies, anti-thrombotics, anti-platelet agents, IIb/IIIa blockers, antiviral agents, anti-cancer agents, chemotherapeutics, thrombolytics, vasodilators, antibiotics, growth factor antagonists, free radical scavengers, radiopaque agents, anti-angiogenesis agents, angiogenesis drugs, cyclooxygenase inhibitors, phosphodiesterase inhibitors, cytokine inhibitors, nitrogen oxide donors, and cytokine activators. Also combinations of drugs can be used.

A coating provided on the intraluminal device in accordance with an embodiment of the present invention may comprise other substances in addition to the therapeutic agent and the triglyceride coating. It is for example possible to add some substances, in particular some natural or synthetic polymeric substances, binders, thickening agents, etc. to the coating in order to stabilise it. The amount of such substances is however preferably kept below 80% by weight, more preferably below 50% by weight and most preferably below 20% by weight of the global coating composition in order to maintain the improved biocompatibility of the transesterified triglyceride coating as much as possible.

In order to control or tailor the release of the therapeutic agent out of the coating, a top coating can be applied on top of this coating, in particular a top coating of the same or a different biocompatible transesterified triglyceride composition. The top coating can however also be of a different chemical composition. The rate at which the therapeutic agent is delivered can further be controlled by the thickness of the coating applied, the ratio of therapeutic agent to transesterified triglycerides in the coating or by providing multiple coatings with varying drug concentrations. In a device according to an embodiment of the present invention the release of therapeutic agent can further be controlled by the selection of an appropriate biocompatible transesterified triglycerides having a certain stability level and melting point or slip melting point.

The transesterified triglyceride coatings of the implantable devices according to an embodiment of the present invention preferably have a melting point lower than 100° C. and more preferably lower than 70° C. so that the therapeutic agent can be mixed with the transesterified triglycerides in the molten state thereof without having a deleterious effect on the therapeutic agent.

Therapeutic agents can also be dissolved in the coating using solvents. A mixture can be made of the therapeutic agent, the transesterified triglyceride mixtures in molten state and a volatile solvent such as, but not limited to acetone, chloroform, dichloromethane, diethylether, ethyl acetate. Ethanol, which forms an emulsion with the transesterified triglyceride mixtures, can also be used.

In a further aspect, the present invention relates to a method for providing an intraluminal device, in particular an intraluminal prosthesis, stent, shunt, catheter or local drug delivery device, with increased biocompatibility which comprises providing the intraluminal device with at least one coating comprising transesterified triglycerides.

According to a particular embodiment of this method, the coating comprising transesterified triglyceride mixtures contains a therapeutic agent comprised in a matrix which sticks to the intraluminal device. In accordance with this aspect of the invention, the matrix is formed by biocompatible transesterified triglyceride mixtures, which comprises said therapeutic agent, and which is applied in a flowable state onto the device.

When the transesterified triglyceride mixtures have a sufficiently low viscosity (after heating), it can be applied in a molten state onto the device. Usually, use is however preferably made of a solvent which is mixed with the transesterified triglycerides before applying the coating onto the device and, after having applied the mixture of solvent and transesterified triglycerides onto the device, the solvent is allowed to evaporate.

When the transesterified triglyceride mixtures are soluble in the solvent, a solution of the transesterified triglycerides in the solvent can first be made after which the therapeutic agent, when not yet comprised in the transesterified triglycerides, can be added. When the transesterified triglycerides are not soluble, a homogeneous mixture is first made, in particular an emulsion. Alternatively, the therapeutic agent can first be dissolved or dispersed in the solvent before mixing it with the transesterified triglycerides.

A method according to a particular embodiment of the present invention comprises the following steps:
a) Cleaning, degreasing and drying of the prosthesis
b) Dipping of the prosthesis in an anti-oxidative solution and drying it, e.g. by air-drying,
c) Making an emulsion or solution of transesterified triglyceride mixtures and a solvent
d) Applying to the prosthesis body, a therapeutic agent containing transesterified triglyceride/solvent emulsion or solution, e.g. using dipcoating or spraycoating or any other coating method,
e) Dry till the solvent is evaporated, e.g. by air-drying.
f) Optionally repeating the previous steps multiple times, eventually using different therapeutic agents.
g) Further dry the prosthesis, e.g. in a sterile laminar airflow until the transesterified triglyceride coating is solidified. This step can also be performed in a vacuum chamber.

Optionally, this method further comprises the step of dissolving one or more therapeutic agents in the emulsion/solution obtained by step (c). The therapeutic substance needs only to be dispersed throughout the solvent/transesterified triglycerides emulsion or solution so that it may be either in a true solution with the solvent/transesterified triglyceride emulsion or solution or dispersed in fine particles in the solvent/transesterified triglyceride emulsion or solution.

The transesterified triglycerides could for example be enriched with EPA and optionally DHA. It is also possible to add alfa-tocopherol and/or a derivative thereof to the transesterified triglycerides. Alternatively, transesterified triglycerides can be selected which comprise groups which are therapeutically active, such as unsaturated fatty acid groups, or a therapeutic agent can be bound to the transesterified triglycerides using any chemical bonding technique. When the transesterified triglycerides are already provided in this way with a therapeutic agent, it is not necessary any more to add a therapeutic agent although it is still possible to add further therapeutic agents.

After drying, a topcoat, comprising biocompatible transesterified triglycerides or composed out of any other coating material can be added by a coating technique such as dip coating, spray-coating or any other equivalent coating method.

After drying, the obtained coated prosthesis can be used as such or further dried and sterilised. Light-protection and vacuum packaging of the obtained coated prosthesis is advisable to maintain the biocompatible characteristics when stored.

The inclusion of biocompatible transesterified triglycerides according to an embodiment of the present invention in intimate contact with a drug covering the prosthesis allows the drug to be retained in the prosthesis in a resilient matrix during expansion of the prosthesis and also slows the administration of drug following implantation.

Any method of the invention can be used whether the prosthesis has a metallic or polymeric surface. Methods of the present invention are also extremely simple since they can be effected by simply immersing the prosthesis into the solution (emulsion) or by spraying the solution (emulsion) onto the prosthesis. The amount of drug to be included onto the prosthesis can be readily controlled by, but is not limited to, using different drug concentrations and/or different coating application methods and/or the amount of solvent that is used. The rate at which the drug is delivered can be controlled by the selection of an appropriate triglyceride combination at a certain stability level and melting point and by the ratio of drug to transesterified triglycerides in the solution. The release rate can be further controlled by using additional barrier coatings or multiple layers of coating with varying drug concentrations. Furthermore this system allows the use of different therapeutic agents. In operation, prosthesis made according to the present invention can deliver drugs to a body lumen by introducing the prosthesis transluminally into a selected portion of the body lumen and radially expanding the prosthesis into contact with the body lumen. The transluminal delivery can be accomplished by a catheter designed for the delivery of the prostheses and the radial expansion can be accomplished by balloon expansion of the prosthesis, by self-expansion of the prosthesis or a combination of self-expansion and balloon expansion.

Thus the present invention provides a prosthesis which may be delivered and expanded in a selected body lumen or conduit without losing a therapeutically significant amount of a drug or gene applied thereto. It also provides a drug or gene containing prosthesis which allows for a sustained release of the drug or gene to luminal or conduit tissue.

The underlying structure of the prosthesis used according to the invention can be virtually any prosthesis design, for example of the self-expanding type or of the balloon expandable type, and of metal or polymeric material. Thus metal prosthesis designs such as those disclosed in U.S. Pat. No. 4,733,665 and U.S. Pat. No. 5,603,721 may be used in the present invention. Also prosthesis with special surface treatments or special designs to optimise local drug delivery are especially suitable for this invention (for example: DE199 16 086 A1, EP 0 950 386 A2, EP 1 132 058 A1, WO 01/66036 A2, WO 98/23228, U.S. Pat. No. 5,902,266, U.S. Pat. No. 5,843,172, the content of which is incorporated by reference). The surface of the prosthesis can in particular be provided with perforating holes or pits which can be filled with the coating material to increase the load of therapeutic agent and/or to slow down the release. After having applied the coating, the surface of the prosthesis next to the holes or pits can be wiped off or cleaned to remove the coating material. The present invention therefore does not only embrace continuous coatings covering the entire prosthesis but also discontinuous local coatings or combinations of local coatings and continuous top coatings applied thereover. The coating further does not need to be applied on the surface of the prosthesis. When using for example porous prostheses, the coating may be located within the pores of the prosthesis. The prosthesis could be made of virtually any biocompatible material having physical properties suitable for the design. For example, tantalum, nitinol, cobalt chromium and stainless steel have been proven suitable for many such designs and could be used in the present invention. Also, prostheses made of biostable or bioabsorbable polymers such as poly(ethylene terephthalate), polyacetal, poly(lactic acid), poly(ethylene oxide)/poly(butylene terephthalate) copolymer and/or bioabsorbable metal alloys could be used in the present invention. Although the prosthesis surface should be clean and free from contaminants that may be introduced during manufacturing, the prosthesis surface requires no particular surface treatment in order to retain the coating applied in the present invention.

In the context of coatings of implantable devices, more particularly endoluminal devices, the transesterified triglycerides chosen should preferably be biocompatible and minimise irritation to the vessel wall when the prosthesis is implanted. The ratio of therapeutic substance to the transesterified triglycerides/solvent solution or emulsion in the solution will depend on the efficacy of the transesterified triglycerides in securing the therapeutic substance onto the prosthesis and the rate at which the coating is to release the therapeutic substance to the tissue of the blood vessel or body conduit. More coating substance may be needed if it has relatively poor efficacy in retaining the therapeutic substance on the prosthesis and more coating substance may be needed in order to provide an elution matrix that limits the elution of a very soluble therapeutic substance. A wide ratio of therapeutic substance to transesterified triglycerides/solvent solution or emulsion could therefore be appropriate, in particular a weight ratio ranging from about 100:1 to 1:100.

According to another aspect, the present invention relates to the use of transesterified triglyceride mixtures to produce micro and macro particles that can contain one or more bioactive substances for dermal, enteral or parenteral delivery of the bioactive substance. Furthermore they can be used to deliver bioactive substances intraluminal, for example intrabronchial, intrauteral, and intrathecal. Also in this embodiment use of top coatings made of similar or different chemical compositions can be used to optimise drug release.

Furthermore use of multiple layers, containing different drug concentrations can be used to optimise drug release.

In one particular embodiment, a (non-chemical) fat transformation process is used, namely fractional crystallisation, also referred to as fractionation or winterizing. Winterizing is the process that makes use of depressed or “winter” temperatures to fractionate or separate an oil into two components: an olein (a liquid fraction with lower melting point than the original oil) and a stearin (a solid fraction with higher melting point). It is envisaged that this can be of interest in the coating of medical devices.

Natural oils, suitable for fractionation, are for example but not limited to coconut oil, palm oil and palm kernel oil.

Palm oil is derived from the flesh of the fruit of the oil palm species E. Guineensis. Palm oil is semi-solid at room temperature; a characteristic brought about by its approx. 50 percent saturation level. Palm oil (and its products) has good resistance to oxidation and heat at prolonged elevated temperatures. In fact, in many instances, palm oil has been used as 100 percent replacement for traditional hydrogenated seed oils such as soybean oil and canola.

Typical fatty acid composition of palm oil is given as:

| | |
|---|---|
| C12:0 Lauric | 0.2% |
| C14:0 Myristic | 1.1% |
| C16:0 Palmitic | 44.0% |
| C18:0 Stearic | 4.5% |
| C18:1 Oleic | 39.2% |
| C18:2 Linoleic | 10.1% |
| Others | 0.9% |

A major advantage of palm oil is that, unlike hydrogenated oils with the same melting point, it contains no trans fatty acids which are now accepted to be risk factors for heart disease.

Palm olein is the liquid fraction obtained by fractionation of palm oil after crystallization at controlled temperatures. The physical characteristics of palm olein differ from those of palm oil. It is fully liquid in warm climate and has a narrow range of glycerides. Its average fatty acid composition is palmitic acid (41%), oleic acid (42%) and linoleic acid (12%).

Palm stearin is the more solid fraction obtained by fractionation of palm oil after crystallization at controlled temperatures. It is thus a co-product of palm olein. Palm stearin is a very useful source of fully natural hard fat component for products such as shortening and pastry and bakery margarines. Its average fatty acid composition is palmitic acid (57%), oleic acid (29%) and linoleic acid (7%)

By varying the fractionation conditions, the relative yields of the two fractions can be changed. Fractionation can be modified to give products of different characteristics, notably palm mid fractions. By fractionation, various grades of olein and stearin are obtainable, enabling to select the grade with the required properties.

Iodine values and melting points of a typical palm oil and palm oil fractions, obtained by fractional crystallisation, are gathered in the table below:

| | | Melting |
|---|---|---|
| fraction | Iodine value | point (° C.) |
| Palm oil | 50-55 | 33-39 |
| Palm olein | 56-62 | 19-24 |
| Palm stearin | 28-45 | 47-54 |

In one embodiment the winterizing procedure can be used so as to provide a coating for implantable devices, for micro and macro particles using the solid fraction. For instance, in view of the properties of palm stearin given above, palm stearin can be used as a coating for implantable devices. According to a further embodiment, the winterizing procedure can be used so as to provide biocompatible coatings using the liquid fraction.

Additionally or alternatively, transesterified triglycerides of the present invention may be subjected to chemical hardening or hydrogenation. Most particularly, hydrogenation may be performed as described by U.S. Pat. No. 6,229,032 and EP-0,917,561 to limit the formation of trans bonds.

Thus, according to one aspect of the present invention, two or three oil transformation processes (hydrogenation, trans- or interesterification, and fractional crystallisation) are combined to obtain an optimal end product, e.g. to transform a raw material with fluctuating composition into a product with nearly constant properties, with regard to biocompatibility, chemical stability and drug release characteristics.

### Example 1

### Chemical Transesterification of Glyceride Mixtures

The following starting materials have been used:

| | Melting |
|---|---|
| triglyceride | point (° C.) |
| Trilinolein | −15.3 |
| Triolein | −4.4 |
| Tristearin | 73.8 |

The following transesterification procedure was followed: Required amounts of tristearin, triolein and trilinolein are put in a vacuum furnace at 70° C. during one hour.
1. 0.01 g NaOMe is added to the starting materials, then the mixture is heated to 110° C. and continuously stirred during one hour.
2. The solution is washed with hot water with separatory funnel.
3. The solution is filtrated over a 0.2 μm filter in a furnace at 80° C.
4. The resulting product is put in a vacuumfurnace at 70° C. during 2 hours.
   Three different transesterification products were made by the above protocol:

| | | | |
|---|---|---|---|
| | | Iodine | |
| | | Value | MP |
| Mixture | Composition | (NMR) | (° C.) |

| | | | | |
|---|---|---|---|---|
| AP054 | tristearin | 30 wt % g | 68.1 | 64-65 |
| | triolein | 30 wt % g | | |
| | trilinolein | 40 wt % g | | |
| AP060 | tristearin | 30 wt % g | 80.4 | 64-65 |
| | triolein | 40 wt % g | | |
| | trilinolein | 30 wt % g | | |
| AP061 | tristearin | 20 wt % g | 90.3 | 64-65 |
| | triolein | 40 wt % g | | |
| | trilinolein | 40 wt % g | | |

The melting points (MP) were determined by DSC (digital scanning calorimetry) as the highest peak in the graph.

The slip melting point of these transesterified triglycerides was also measured and was about 62° C.

### Example 2

### Stents Coated with Transesterified Triglycerides

The transesterification product AP060 from Example 1 was used to coat stents by dipcoating according to the following procedure:
1. Transesterified triglycerides were mixed with 450 vol. % acetone, heated to 55° C. and continuously stirred until a homogeneous solution was obtained.
2. Sterile stents (Nexus II, 3.5×15 mm, Occam International BV (Biosensors), Eindhoven, The Netherlands), mounted on balloon catheters, were dipped in the homogeneous solution during 5 seconds.
3. The solvent (acetone) was allowed to evaporate at room temperature.

Light optical microscope analysis was performed of stents with a transesterified triglyceride coating (different magnifications) and of uncoated stents in comparison. It was determined that for the transesterified triglyceride coated stents, a very thin and homogeneous coating was obtained using the above protocol.

After coating, the stents were also expanded. This was performed at room temperature, which is a worst case situation: usually, stents are expanded at 37° C. which makes the coating more soft.

This example demonstrates that a stent can be coated with a transesterified triglyceride coating. The transesterified triglyceride coating does not start flaking upon stent expansion.

### Example 3

### Scratch Test on Transesterified Triglyceride Coating

Stainless steel plates (316 LVM), having a total surface area of 1 cm², were dipped into the solutions, made in Example 2. After dipping, the plates were allowed to cool down to room temperature. A manual scratch was made with a syringe needle. This scratch was examined by light optical microscopy.

It was determined that the coating material was pushed aside when the needle scratched into the coating. Almost all material remained on the plates. No delaminations or flakes were observed.

### Example 4

### Biocompatibility Test of Transesterified Triglyceride Coating

Transesterified triglycerides (lot. No. AP060—see Example 1) were heated to 55° C. Acetone was added while continuously stirring until a homogeneous solution was obtained. 4 stainless steel stents were dipped into the solution followed by air-drying.

As a control, 2 stents were dipcoated coated with hydrogenated triglycerides by dipping them into a solution containing hydrogenated triglycerides and acetone at 50° C.

Stents (3.0×15 mm and 3.5×15 mm) were randomly implanted in the coronary arteries of pigs. Animals were sacrificed 7 days after stent implantation. All stented vessels were patent.

### Histopathology

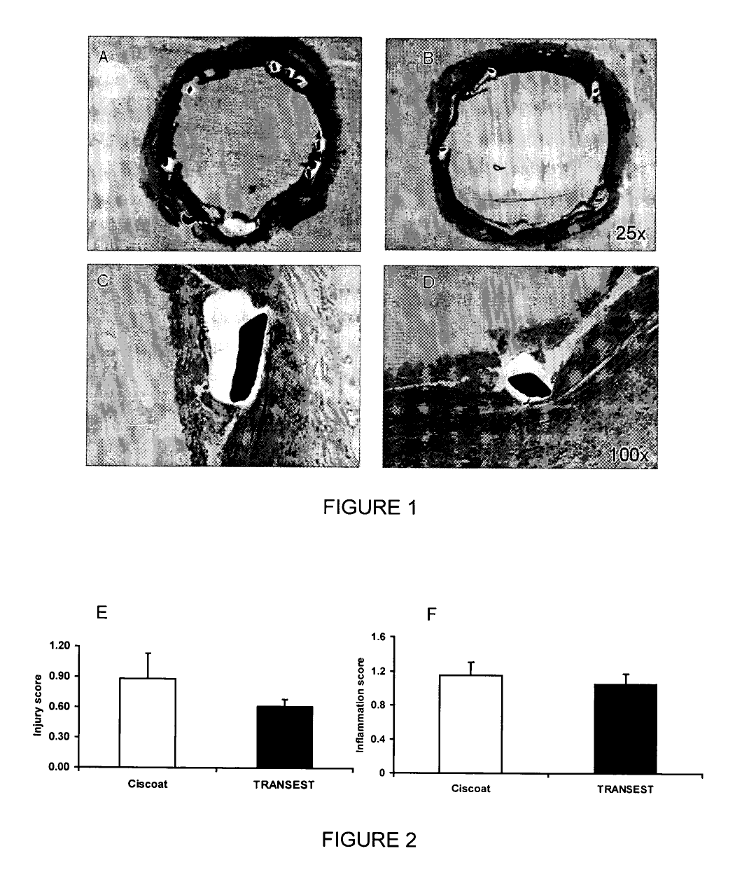

In the vessels with a stent coated with transesterified triglycerides (TRANSEST), the media was deeply compressed but only a few internal elastic lamina were lacerated and the injury induced by stent implantation was moderate (FIG. 1; B, D and FIG. 2 E).

The inflammatory response of the TRANSEST stent groups was low, only one stent strut was surrounded by inflammatory cells (FIG. 1; B, D and FIG. 2 F).
Arterial injury at each strut site was determined by the anatomic structures penetrated by each strut. A numeric value from 0 (no injury) to 3 (most injury) was assigned.
0=internal elastic lamina (IEL) and media intact or media is compressed but <50%;
1=IEL lacerated, or media is compressed but >50%;
2=IEL and media lacerated, external elastic lamina (EEL) intact;
3=IEL, media and EEL lacerated
The inflammatory reaction at every stent filament site was carefully examined searching for inflammatory cells, and scored as followed:
1=sparsely located histiolymphocytic infiltrate around the stent filament;
2=more densely located histiolymphocytic infiltrate covering the stent filament, but no foreign body granuloma or giant cells;
3=diffusely located inflammatory cells and/or giant cells, also invading the media.
No severe thrombosis formation was observed around the stent struts.

In the vessels with stents coated with hydrogenated triglycerides (Ciscoat), the media was deeply compressed and some of the internal elastic lamina was lacerated and the injury induced by stent implantation was higher than the TRANSEST stent (0.88±0.25 vs.0.61±0.07; FIG. 1; A, C and FIG. 2; E), and it resulted in a higher inflammatory score (1.16±0.07 vs. 1.05±0.12; FIG. 1; A, C and FIG. 2; F).

### Conclusion:

This study showed a biocompatible performance of the stents coated with transesterified triglycerides 7 days post stent implantation which is comparable to the hydrogenated triglycerides control group.

### Example 5

### Drug Release with an Enzymatically Transesterified Triglyceride Coating

Transesterified triglyceride mixtures were made using the following procedure:
30 weight % tristearin, 30 weight % triolein, 40 weight % trilinolein are put in a vacuum furnace at 70° C. during 1 hour
the mixture is stirred during 30 minutes at 75° C.
1% of 1,3 specific lipase (lipozyme) is added to the mixture at 75° C. during 1 hour.
Filtration over Whatman no. 4 filter.
   The slip melting point of the resulting mixture was 60° C.

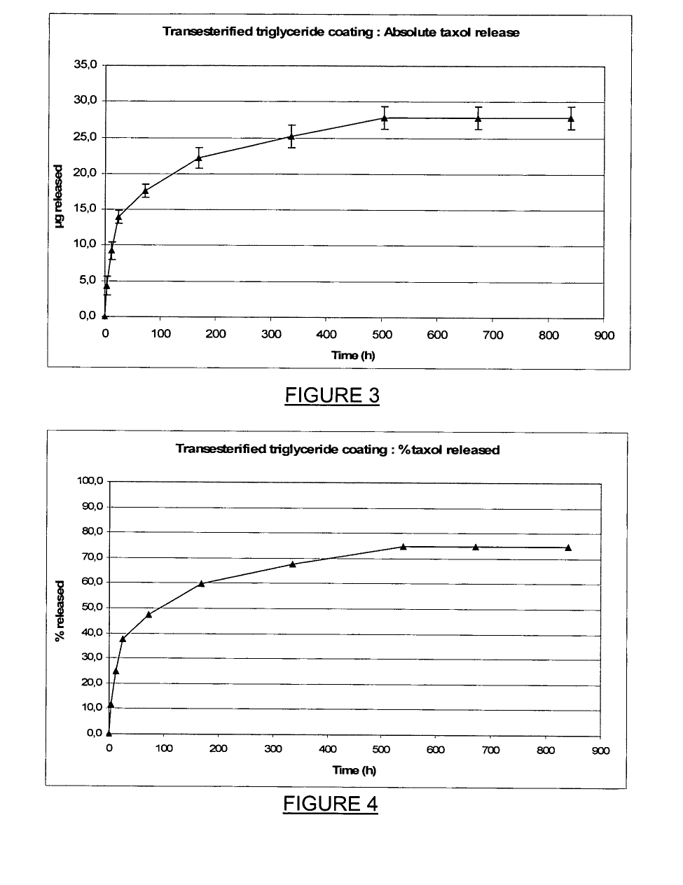

5 weight % of paclitaxel (taxol) and acetone were added to this triglyceride mixture, heated to 50° C. and stirred until homogeneous. Stainless steel samples were dipped into the solution followed by air-drying. The samples were put into 5 ml of release medium (PBS with 0.02% (w/v) sodium azide) at 37° C. and continuously shaken with a rocking table. At different time points, samples were put in a new vial with fresh release medium. The medium of the previous vials were analyzed with HPLC to determine the concentration of paclitaxel that was released. After the test, the remaining paclitaxel was liberated from the coating on the stainless steel samples by dissolving the coating in acetone at 50° C. Cumulative and relative release curves are respectively shown in FIG. 3 and FIG. 4.

As shown in FIG. 4, after 5 weeks, 75% of the drug has been released. However, in an in vivo situation, the remaining 25% will also be released because the triglycerides will be metabolized by the smooth muscle cells of the vascular wall. This example proves that drugs can be mixed with transesterified triglycerides and that transesterified triglycerides have drug release capabilities.

### Example 6

### Chemical Transesterification

In this example, a blend (mixture) was made from only two components: tristearin (70 weight %) and triolein (30 weight %). Slip melting points (SMP) and HPLC diagrams were measured for the starting blend (comparative, TR031 in the table below) and for the transesterified fatty acid triglyceride mixture, obtained after 2 hours reaction time in the presence of 0.05 g NaMeO (TR032 in the table below).

| | | | | Reaction | SMP |
|---|---|---|---|---|---|
| Mixture | Composition | | | time | (° C.) |
| TR031 | tristearin | 70 wt % | 1.8906 g | No reaction | 70 |
| | triolein | 30 wt % | 0.8109 g | | |
| TR032 | tristearin | 70 wt % | 1.8912 g | 2 hours | 62 |
| | triolein | 30 wt % | 0.8109 g | | |

HPLC measurements were performed according to AOCS Official Method Ce 5b-89.

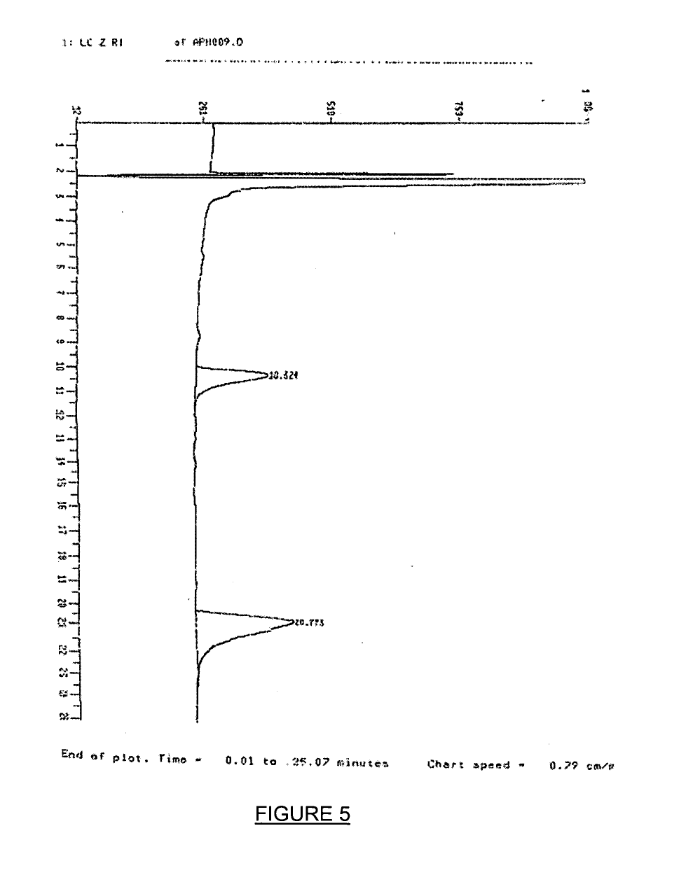

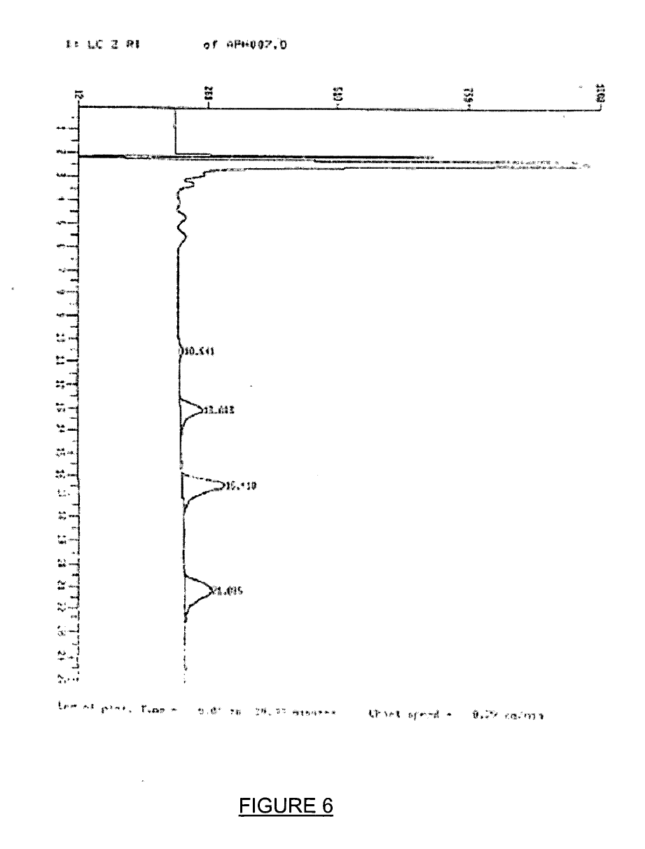

In the HPLC refractive index diagram of the comparative blend TR031 (FIG. 5), only two peaks are visible, corresponding with the two starting components (OOO, i.e. triolein with a retention time around 10.3 minutes, and SSS, i.e. tristearin with a retention time around 21 minutes). Peak areas were also calculated—see table below.

| Retention | Peak area | Relative |
|---|---|---|
| time (min) | (arbitrary units) | percentage (%) |
| 10.324 | 4796 | 31.3 |
| 20.773 | 10509 | 68.7 |

In the HPLC refractive index diagram of TR032 (FIG. 6), apart from the peaks corresponding with the two basic components (OOO and SSS), two extra peaks are visible, namely OOS, SOO and OSO (i.e. dioleylstearylglycerol) for the peak with retention time approximately 13 min. and SOS, OSS, SSO (i.e. oleyidistearylglycerol) for the peak with retention time 16.4. Peak areas were calculated—see table below. This proves that a transesterification reaction has taken place.

| Retention | | Relative |
|---|---|---|
| time (minutes) | Peak area | percentage (%) |
| 10.541 | 243.01 | 2.9 |
| 13.063 | 1657 | 19.9 |
| 16.418 | 3784 | 45.4 |
| 21.085 | 2646 | 31.8 |

1-22. (canceled)

23. A bio-compatible coating composition for a medical device comprising transesterified triglycerides, said transesterified triglycerides consisting of a mixture of fatty acid triglycerides, wherein the slip melting point of said mixture is above 50° C., the fatty acid composition of said mixture comprises at least two different fatty acids and consists of from 20 to 95% by weight of saturated fatty acids and 80 to 5% by weight of unsaturated fatty acids, and wherein at least one fatty acid triglyceride (a1) from said mixture bears the same saturated fatty acid residue on each position of the glycerol moiety and corresponds to at least 10% by weight of said mixture.

24. A bio-compatible coating composition according to claim 23, wherein said composition further comprises at least one biologically active compound.

25. A bio-compatible coating composition according to claim 23, wherein the content of said bioactive compound is from 0.01% to 50% by weight of said coating composition.

26. A bio-compatible coating composition according to claim 23, wherein said active compound is selected from the group consisting of immunosuppressants, anti-inflammatories, anti-proliferatives, anti-migratory agents, anti-fibrotic agents, proapoptotics, calcium channel blockers, anti-neoplastics, antibodies, anti-thrombotics, anti-platelet agents, IIb/IIIa blockers, antiviral agents, anti-cancer agents, chemotherapeutics, thrombolytics, vasodilators, antibiotics, growth factor antagonists, free radical scavengers, radiopaque agents, anti-angiogenesis agents, angiogenesis drugs, cyclooxygenase inhibitors, phosphodiesterase inhibitors, cytokine inhibitors, nitrogen oxide donors, and cytokine activators.

27. A bio-compatible coating composition according to claim 23, wherein the slip melting point of said mixture is below 70° C.

28. A bio-compatible coating composition according to claim 23, wherein the fatty acid composition of said mixture consists of linoleic acid, oleic acid and stearic acid.

29. A bio-compatible coating composition according to claim 23, wherein the fatty acid chains composition of said mixture consist of between 20-40% linoleic acid, between 20-40% stearic acid and between 20-40% oleic acid.

30. A medical device provided with at least one coating having a coating composition according to claim 23.

31. A medical device according to claim 30, wherein said device is selected from the group consisting of an intraluminal prosthesis, a stent, a shunt, a catheter, a local drug delivery device, surgical wires or clips.

32. A medical device provided with at least one coating having a coating composition according to claim 28.

33. A medical device according to claim 32, wherein said device is selected from the group consisting of an intraluminal prosthesis, a stent, a shunt, a catheter, a local drug delivery device, surgical wires or clips.

34. A medical device provided with at least one coating having a coating composition according to claim 29.

35. A medical device according to claim 34, wherein said device is selected from the group consisting of an intraluminal prosthesis, a stent, a shunt, a catheter, a local drug delivery device, surgical wires or clips.

36. A medical device provided with at least one coating having a coating composition according to claim 27.

37. A medical device according to claim 36, wherein said device is selected from the group consisting of an intraluminal prosthesis, a stent, a shunt, a catheter, a local drug delivery device, surgical wires or clips.

38. A method for producing a medical device according to claim 23, comprising the steps of:
providing a medical device,
coating said medical device with a coating having a coating composition according to claim 1.

39. A method according to claim 38, wherein the coating step is realised by dipping said medical device in a solution of said coating composition in a solvent or by spraying said coating composition on said medical device.

40. A mixture of fatty acid triglycerides, wherein the slip melting point of said mixture is above 50° C., the fatty acid composition of said mixture comprises at least three different fatty acids and consists of from 20 to 95% by weight of saturated fatty acids and 80 to 5% by weight of unsaturated fatty acids, and wherein at least one fatty acid triglyceride (a1) from said mixture bears the same saturated fatty acid residue on each position of the glycerol moiety and corresponds to at least 10% by weight of said mixture.

41. A mixture of fatty acid triglycerides according to claim 40, wherein the slip melting point of the mixture is below 70° C.

42. A mixture of fatty acid triglycerides according to claim 40 wherein the fatty acid composition of said mixture consists of linoleic acid, oleic acid and stearic acid.

43. A mixture of fatty acid triglycerides according to claim 40, wherein the fatty acid chains composition of said mixture consist of between 20-40% linoleic acid, between 20-40% stearic acid and between 20-40% oleic acid.
